(19) United States
(12) Patent Application Publication
(10) Pub. No.: US 2004/0071739 A1
(43) Pub. Date: Apr. 15, 2004

(54) Methods of preparing an anti-tumor vaccine

(75) Inventor: Allan M. Green, Cambridge, MA (US)

Correspondence Address: LAHIVE & COCKFIELD, LLP., 28 STATE STREET, BOSTON, MA, 02109, US

(73) Assignee: Select Therapeutics, Inc., Ottawa, ON (CA)

(21) Appl. No.: 10/397,067

(22) Filed: Mar. 25, 2003

Related U.S. Application Data
(63) Continuation of application No. 09/713,566, filed on Nov. 15, 2000, now abandoned.
(60) Provisional application No. 60/165,541, filed on Nov. 15, 1999.

(51) Int. Cl. A61K 39/00

(52) U.S. Cl. 424/277.1

(57) ABSTRACT

A method of preparing an autologous anti-tumor vaccine by chemically linking a tumor or anti-microbial extract with a shiga-like toxin B fragment.

### RELATED APPLICATIONS

This application claims priority to U.S. Provisional Application Serial No. 60/165,541, “Verotoxin B Subunit for Immunization,” filed on Nov. 15, 1999. This application is also related to U.S. application Ser. No. 09/312;338, entitled “Verotoxin B Subunit for Immunization” filed on May 14, 1999, the entire contents of both of which are hereby incorporated herein by reference.

### BACKGROUND OF THE INVENTION

Cancer is the second leading cause of death in the United States accounting for almost 500,000 deaths each year. More than 1,000,000 new cases of cancer are diagnosed in the United States annually. The incident of cancer is increasing largely as a byproduct of the greater lifespan of the aging population. Cancer is a leading cause of death in all industrialized nations, where life expectancy continues to increase. It is expected that cancer morbidity and mortality will continue to increase in all industrialized areas of the world.

Cancer of the lung is the most common malignancy in the U.S. Neoplasms of the lung and gastrointestinal tract account for 30% of all malignancies. There are over 160,000 new cases of lung cancer annually in the U.S. with 143,000 annual fatalities. Seventy percent of lung cancer patients die of recurrent tumor following “curative” surgery. The five year survival following diagnosis is 10%.

Colorectal cancer is currently the second most commonly occurring cancer in the United States. The present incidence of colorectal cancer in the U.S. is about 150,000 cases new cases per year with a mortality approaching 60,000 per year. The five year survival following diagnosis is 50%.

Melanoma strikes about 32,000 patients per year in the U.S. The incidence of melanoma is dramatically increasing and by the year 2000, 1 of every 100 Americans will develop melanoma at some point in their lifetime.

Other forms of cancer, including ovarian, pancreatic, and especially breast continue to be major causes of cancer related mortality.

Almost all forms of cancer continue to be refractory to treatment despite many years of therapeutic experience. Vaccine development has been slow and no vaccine currently exists for any form of cancer. There is a continuing need for the development of new therapeutic and prophylactic compounds efficacious in the prevention and treatment of all forms of cancer.

### SUMMARY OF THE INVENTION

In one embodiment, the invention pertains to a method of preparing an autologous anti-tumor vaccine comprising chemically linking a tumor extract with a shiga-like toxin B fragment. Advantageously, the chemical linkage between the tumor extract and the shiga-like toxin B fragments is a carbodimide or a cyanogen bromide linkage.

In another embodiment, the invention relates to a method for preparing an anti-pathogen vaccine, by chemically linking an anti-pathogenic extract with a shiga-like toxin B fragment. The extract may be linked to the Shiga-like toxin through a carbodiimide or a cyanogen bromide linkage.

The invention also pertains to a method of activating autologous antigen presenting cells, comprising contacting said cells with the vaccines of the invention. The cells can be contacted with the vaccine in vivo (e.g., administered to a patient, e.g., parentally) or in vitro. Preferably, the cells are dendritic cells.

### DETAILED DESCRIPTION OF THE INVENTION

The B fragment of Shiga-Like Toxin (SLT or verotoxin) binds to the surface of dendritic cells by associating with the CD77 and CD19 surface molecules. Dendritic cells are the paradigmatic antigen-presenting cell (APC) which take up and process exogenous antigens and, then, “present” the antigens on their surface in the context of the major histocompatibility molecules (MHC's) required to activate the T-cell effectors of the cellular arm of the immune system. Presentation of antigens by such antigen-presenting cells in the context of the MHC's is essential in triggering antitumor and anti-viral T cell responses.

Previous work has demonstrated that SLT B fragments expressed as chimeric molecules bearing tumor antigens efficiently carry the associated antigens into dendritic cells and B lymphoblastoid cells and direct the antigens retrograde into the antigen-processing pathway for presentation in association with the MHC's.

Tumor antigens presented to T cells by dendritic cells in such a manner may trigger effective anti-tumor responses. Johannes and Tartour at the Curie Institut in Paris have shown regression of PC12 mastocytome in rodents using such a system.

The use of molecular biology to create chimeric molecules combining the SLTB fragment with the entire repertoire of tumor associated antigens would be complex, time-consuming and costly. Further, there is little assurance that any given patient's tumor antigens would be fully and effectively represented by such an artificial mix of chimeric molecules.

Recent work has shown that in vitro co-culture and activation of autologous dendritic cells with autologous melanoma tumor extract activate the dendritic cells and initiate and anti-tumor response after re-injection. This work suggests that more efficient means of activating dendritic cells might lead to even more effective anti-cancer treatments.

The present invention presents an technique for the creation of autologous anticancer and anti microbial vaccines by:

Extraction of autologous tumor antigens from a patient's primary or metastatic tumor;

Covalent or non-covalent linkage of the tumor extract to the B chain of shiga like toxin (SLT);

Incubation of the linked tumor antigen/B chain with dendritic cells in vitro. The invention also contemplates injection of the linked tumor antigen/B chain into the patient subcutaneously, intradermally, intramuscularly or directly into one or more lymph nodes.

Linkage of tumor extract and B chain of SLT may be performed by a number of techniques well known to those skilled in the art of biochemistry. Preferred techniques include use of carbodiimide linkage or use of cyanogen bromide activation and linkage. Other standard techniques, such as those described by Cuatrecasas for the creation of affinity chromatography columns, may be adapted for this use. The only requirement is that the techniques allow the final preparation of a pharmaceutically compatible preparation for in vitro use or for parenteral use in subjects. When these preparations are used parenterally, subjects may be pretreated either systemically or locally with cytokines which mobilize antigen-presenting cells such as dendritic cells. GM-CSF is one preferred cytokine for such pretreatment.

The term “antigen” includes agents which provoke an immune response independently and those which are provoke an immune response when incorporated in to a vaccine of the invention. The term “antigen epitope” includes fragments of proteins capable of determining antigenicity. An epitope may comprise, for example, a peptide of six to eight residues in length (Berzofsky, J. and I. Berkower, (1993) in Paul, W., Ed., Fundamental Immunology, Raven Press, N.Y., p.246). Some epitopes may be significantly larger.

For example, antigens include proteins and other molecules which are specifically associated with surfaces of particular types of cancer cells, e.g. tumor cells. Many forms of cancer can be characterized by production of proteins associated with that form of the disease, and are not found in normal tissue. Often these proteins are used at a specific stage of embryonic development, and are not observed during normal adult lifetime. These antigens are particularly useful as a source of epitopes for anticancer vaccines. Examples of tumor antigens include those corresponding to cancers affecting the breast, ovarian, lung, skin, and brain. For example, breast tumors may be characterized by abnormally expressed receptors, e.g. those of the human-EGF-like receptor family (HER). Additionally, the nestin protein, which is expressed by neuroepithelial stem cells during normal mammalian fetal development, is also expressed on tumors of the central nervous system, including most forms of brain cancer (McKay, D. G. Ronald, U.S. Pat. No. 5,338,839, Aug. 16, 1994).

Other examples of tumors expressing antigens contemplated by the present invention include Wilm's tumor (A. J. Buckler, K. M. Call, T. M. Glaser, D. A. Haber, D. E. Housman, C. Y. Ito, J. Pelletier, Rose, E. A. Rose, U.S. Pat. No. 5,350,840), gastrointestinal cancer (R. Fishel et al., International Application WO 95/14085, 05/26/95), cancers characterized by development of multiple drug resistance during chemotherapy (J. M. Croop et al., U.S. Pat. No. 5,198,344), and cancers characterized by the presence of at least one of a large number of oncogenes well known to the skilled artisan, such as Rb, ras, and c-myc, the sequences of which are available for analysis to those with skill in the art.

Alternatively, antigens of the invention may be associated with the surfaces or secretion products of micro-organisms or pathogens. The term “pathogen” is meant to include organisms that cause disorders, such disorders produced by one or more particular species of bacteria, viruses, fungi, and protozoans which are disease-producing organisms. Examples of pathogens include gram-negative bacterial species such as Escherichia coli serotype 0157:H7, Helicobacter pylori, H. mustelae, Haemophilus influenzae and H. ducreyi, Pseudomonas aeruginosa, Shigella dysenteria, Salmonella typhi and S. paratyphi; Gram-positive bacterial species such as Mycobacterium tuberculosis, M. leprae, Clostridium tetani, Staphylococcus aureus, and Streptococcus hemolyticus; obligate intracellular bacterial organisms such as Rickettsia and Chlamydia species; retroviruses, which are RNA containing viruses that use reverse transcriptase to synthesize complementary DNA, including but not limited to HIV-1, and -2; other pathogenic viruses such HSV-I and -II, non-A non-B non-C hepatitis virus, pox viruses, and rabies viruses; fungi such as Candida and Aspergillus species; protozoa such as Cryptosporidium parvum, Entamoeba histolytica and Giardia lamblia; and animal pathogens such as Newcastle disease virus. Obtaining unique epitopes from these organisms by screening proteins and by assaying peptides in vitro are commonly known to those skilled in the art; many examples have been described and the appropriate amino acid residue sequence may be accessed from Genbank.

The term “antipathogenic extract” includes an extract from a pathogen or microorganism which contains antigens which can be used in the methods of the invention to make antipathogenic vaccines. In one embodiment, the antipathogenic extract includes surface proteins or secretion products of the pathogen.

The term “shiga-like toxin” includes cytotoxins similar in structure and function to shiga toxin as well as shiga toxins. The term includes verotoxins which, based upon structural similarity to shiga toxins by sequencing of relevant genes, are often referred to as shiga-like toxins (SLTs). Known SLTs (verotoxins) include SLT-I (verotoxin1), SLTII (verotoxin 2), and SLTIII. Variants of SLTII (isolated and distinguished seriologically on the basis of gene sequence or host specificity) include verotoxin 2c, verotoxin 2e, SLTII, vtx2ha; SLTIIvh, vtx2hb, SLTIIc, SLTIIvp, etc. The term also encompasses the presently unknown SLTs or variants thereof that may be discovered in the future, since their characterization as an SLT or variant thereof will be readily determinable by persons skilled in the art.

Shiga-toxin is a bacterial protein toxin of the AB₅ subunit family that is secreted by Shigella dysenteriae. The A-subunit inhibits protein biosynthesis in higher eukaryotic cells after transfer into the cytoplasm by modifying a conserved residue of 28S rRNA. The B-subunit, a homopentamer (%-B fragments) is responsible for toxin binding to and internalization into target cells by interacting with the glycolipid Gb₃ found in the plasma membrane of these cells. The B-fragment is not toxic but conserves the intracellular transport characteristics of the holotoxin which in many Gb₃ expressing cells is transported in a retrograde fashion from the plasma membrane via endosomes into the biosynthetic/secretory pathway.

The term “mammal” includes warm blooded animals such as, for example, rodents (e.g. rats, mice, hamsters, squirrels), horses, cows, pigs, sheep, cats, dogs, bears, goats, and primates (e.g., monkeys, chimpanzees, gorillas, and, preferably, humans).

The term “antigen-presenting cells” include those cells which display antigens or antigenic fragments. Examples of antigen-presenting cells include some peripheral blood mononuclear cells and, preferably, dendritic cells, e.g., Langerhans cells.

The term “dendritic cells” include Langerhans cells, interstitial dendritic cells, interdigitating dendritic cells, follicular dendritic cells and circulating dendritic cells. Langerhans cells are found in the epidermis and mucous membranes. Interstitial dendritic cells populate most organs such as the heart, lungs, liver, kidney, and gastrointestinal tract. Interdigiting dendritic cells are present in T-cell areas of the secondary lymphoid tissue and the thymic medulla. Circulating dendritic cells include “veiled cells” which constitute about 0.1% of the blood leukocytes.

In general, dendritic cells are covered with a maze of long membrane processes resembling dendrites of nerve cells. Due to their long dendritic processes, dendritic cells have been challenging to study using conventional procedures for isolating lymphocytes and accessory immune-system cells. Dendritic cells tend to express high levels of both class II MHC molecules and the co-stimulatory B7 molecule. For this reason, they are more potent antigen-presenting cells than macrophages and B cells, both of which need to be activated before they can function as APCs. After capturing an antigen in the tissues by phagocytosis or by endocytosis, dendritic cells migrate into the blood of lymph and circulate to various lymphoid organs where they present the antigen to T lymphocytes.

The term “administering” includes routes of administration which allow the vaccine or other composition of the invention to perform its intended function, e.g. stimulate an immune response. Preferred routes of administration include, but are not limited to, orally, intrabronchially, and transdermally. Depending on the route of administration, the vaccine of the invention can be coated with or disposed in a selected material to protect it from natural conditions which may detrimentally effect its ability to perform its intended function. The vaccine of the invention can be administered alone or with a pharmaceutically acceptable carrier. Further, the vaccine or other composition of the invention can be administered as a mixture, which also can be coadministered with a pharmaceutically acceptable carrier.

In another embodiment, the invention features a pharmaceutical composition which includes a composition of the invention and a pharmaceutically acceptable carrier. The phrase “pharmaceutically acceptable carrier” as used herein means a pharmaceutically acceptable material, composition or vehicle, such as a liquid or solid filler, diluent, excipient, solvent or encapsulating material, involved in carrying or transporting a compound(s) of the present invention within or to the subject such that it can performs its intended function. Typically, such compounds are carried or transported from one organ, or portion of the body, to another organ, or portion of the body. Each carrier must be “acceptable” in the sense of being compatible with the other ingredients of the formulation and not injurious to the patient.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

While it is possible for a compound of the present invention to be administered alone, it is preferable to administer the compound as a pharmaceutical composition. Preferred pharmaceutical compositions include those suitable for administration orally, transdermally, or intrabronchially.

The contents of all references, patent applications, patents, and published patent applications cited throughout this application are hereby incorporated by reference.

1. A method of preparing an autologous anti-tumor vaccine comprising chemically linking a tumor extract with a shiga-like toxin B fragment, such that an autologous anti-tumour vaccine is prepared.

2. The method of claim 1, wherein said tumor extract is chemically linked to the shiga-like toxin B fragment through a carbodiimide linkage.

3. The method of claim 1, wherein said tumor extract is chemically linked to the shiga-like toxin B fragment through a cyanogen bromide chemical linkage.

4. A method of activating autologous antigen presenting cells, comprising contacting said cells with a vaccine of claim 1, such that said cells are activated.

5. The method of claim 4, wherein said cells are dendritic cells.

6. The method of claim 4, wherein said vaccine is administered to a patient.

7. The method of claim 6, wherein said vaccine is administered parentally.

8. A method for preparing an antipathogenic vaccine, comprising chemically linking an anti-pathogen extract in with a shiga-like toxin B fragment, such that an antipathogenic vaccine is prepared.

9. The method of claim 8, wherein said antipathogenic extract is chemically linked to the shiga-like toxin B fragment through a carbodiimide linkage.

10. The method of claim 8, wherein said antipathogenic extract is chemically linked to the shiga-like toxin B fragment through a cyanogen bromide linkage.

11. A method for the activation of autologous antigen presenting cells, comprising incubating said cells in vitro with the vaccine of claim 8, such that said cells are activated.

12. The method of claim 11, wherein said cells are dendritic cells.

13. The method of claim 8, wherein said vaccine is administered to a patient.

14. The method of claim 8, wherein said vaccine is administered parentally.
